(19) United States
(12) Patent Application Publication
(10) Pub. No.: US 2016/0106933 A1
(43) Pub. Date: Apr. 21, 2016

(54) SELF-DESTRUCTIVE TYPE SAFE MEDICAL NEEDLE

(71) Applicant: Jiangsu Caina Medical Technology Co., Ltd., Jiangyin (CN)

(72) Inventors: Jun Lu, Jiangyin (CN); Lijie Zhao, Jiangyin (CN); Lin Ni, Jiangyin (CN); Fulin Nie, Jiangyin (CN)

(73) Assignee: Jiangsu Caina Medical Technology Co., Ltd., Jiangyin (CN)

(21) Appl. No.: 14/535,327

(22) Filed: Nov. 7, 2014

(30) Foreign Application Priority Data
Oct. 15, 2014 (CN) 201410546740.5

(51) Int. Cl. A61M 5/50; A61M 5/162

(52) U.S. Cl. CPC A61M 5/50; A61M 5/1626

(57) ABSTRACT

A self-destructive type safe medical needle comprises: a sleeve with an inner cavity, a needle base which can do reciprocating motion in the inner cavity and a needle head, a lock catch head buckled with the needle base, a lock catch head buckled with the needle base, and a conduit connected with the inside of the lock catch head, wherein the sleeve is provided with a barrier block and a lock catch block in the inner cavity, and the needle base is provided with a limiting part which is propped against and matched with the barrier block and a deformable locking part which is locked and matched with the lock catch block. The needle avoids cross infection between patients, secondary use of needle and puncture of staff after use or in the process of destroying. Further, this needle also can realize self-destruction and prevent man-made rotation of the medical needle.

### TECHNICAL FIELD

The invention relates to a self-destructive type safe medical needle.

### BACKGROUND ART

Medical needle, used in infusion or blood collection, is a common medical appliance. Because the ordinary medical infusion needle can be repeatedly used, and if it fails to be effectively cleaned and disinfected, it may be easy to cause cross infection between the patients. In order to solve this problem, disposable infusion needle has been widely used, but this kind of disposable infusion needle is only required to achieve one-time use in the aspect of management system. Meanwhile, in order to avoid secondary use of the infusion needle or puncture of working staff after use or in the process of destroying, and causing the serious consequence of viral cross infection, safe infusion needle has been widely used in medical field. Medical regulations in many countries expressly stipulate that safe infusion needle must be used. In recent years, people have developed many types of disposable safe infusion needles with automatic destroying devices, which can ensure the function of one-time use only from the technical level, but they have complicated structures and high costs.

### SUMMARY OF THE INVENTION

The purpose of this invention is to provide a self-destructive type safe medical needle, so as to improve security, realize self-destruction and avoid secondary use; the invention has simple structure and low cost.

Technical scheme of the invention is as follows: a self-destructive type safe medical needle, comprising a sleeve with an inner cavity, a needle base which can do reciprocating motion in the inner cavity and has a needle head, a lock catch head buckled with the needle base, a lock catch head buckled with the needle base, and a conduit connected with the inside of the lock catch head; the sleeve is provided with a barrier block and a lock catch block in the inner cavity, and the needle base is correspondingly provided with a limiting part which is propped against and matched with the barrier block and a deformable locking part which is locked and matched with the lock catch block.

On the basis of the technical scheme above, this invention further comprises the following attached technical schemes:

The barrier block prevents continuous motion of the needle base, and the lock catch block prevents reverse motion of the needle base.

The locking part is a pair of expansion arms which can be expanded elastically, the tail end of the expansion arm is propped and contacted with the plane of the lock catch block, and the barrier block and the limiting part are in plane propping contact.

The sleeve further comprises a pair of lock catch windows set on one end and butterfly wings set on the other end.

The lock catch head is buckled with the lock catch window, the lock catch head comprises a pair of buckling arms which are buckled with the corresponding lock catch windows and can be deformed elastically, and a through hole which is connected with the conduit.

The sleeve is a hollow sleeve with one open end and one semi-open end, the barrier block is arranged between the open and the lock catch block, and an inclined plane which allows the expansion arms pass through and makes the expansion arms gradually shrink is arranged between the barrier block and the lock catch block.

The limiting part is arranged between the needle head and the locking part, the tail end of the buckling arm is provided with a convex part which is extending outwards, and is buckled on the inner wall of the lock catch window in compressive deformation, and a gap in elastic deformation is allowable between the buckling arm and the lock catch head.

The section of the inner cavity is polygonal, elliptic or partially arc-shaped, and the section of the needle base is circular, elliptic, polygonal or partially arc-shaped, which can prevent man-made rotation in operation.

The advantages of this invention are as follows:

The invention avoids the cross infection between patients, it also avoids secondary use of medical needle and puncture of working staff after use or in the process of destroying, at the same time, this invention also can realize self-destruction and prevent man-made rotation of the medical needle.

### BRIEF DESCRIPTION OF THE DRAWINGS

This invention can be further described by combining with figures and embodiments:

FIG. 1 is the stereoscopic schematic diagram of this invention in the first state;

FIG. 2 is the sectional view of FIG. 1;

FIG. 3 is the stereoscopic schematic diagram of this invention in the second state;

FIG. 4 is the sectional view of FIG. 3.

### DETAILED DESCRIPTIONS OF THE PREFERRED EMBODIMENTS

### Embodiment

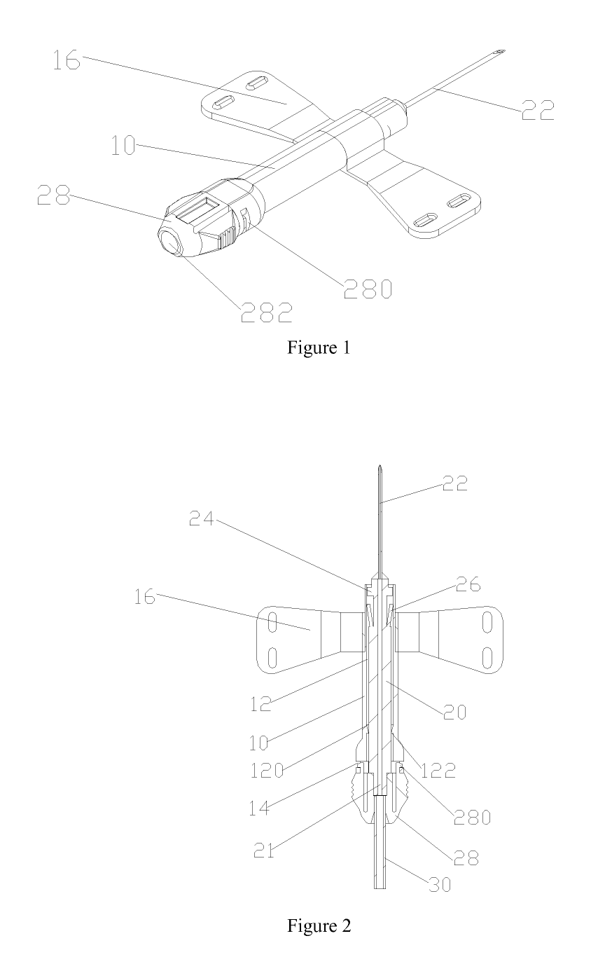

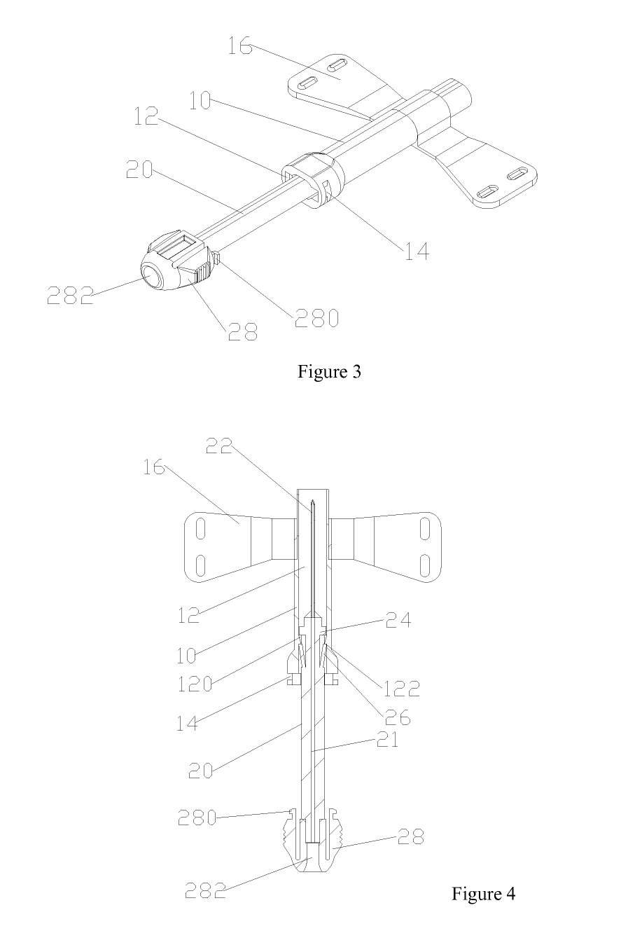

As shown in FIGS. 1-4, this invention provides a specific embodiment of self-destructive type safe medical needle, wherein it comprises: a sleeve 10 with an inner cavity 12, a needle base 20 which can do reciprocating motion in the inner cavity 12 and has a needle head 22, a lock catch head 28 buckled with the needle base 20, and a conduit 30 connected with the inside of the lock catch head 28; the sleeve 10 is provided with a barrier block 120 and a lock catch block 122 in the inner cavity 12, and the needle base 20 is correspondingly provided with a limiting part 24 which is propped against and matched with the barrier block 120 and a deformable locking part 26 which is locked and matched with the lock catch block 122. The barrier block 120 prevents continuous motion of the needle base 20, and the lock catch block 122 prevents reverse motion of the needle base 20. The locking part 26 is preferably a pair of expansion arms which can be expanded elastically, the tail end of the expansion arm is propped and contacted with the plane of the lock catch block 122, and the barrier block 120 and the limiting part 24 are in plane propping contact.

The sleeve 10 is a hollow sleeve with one open end and one semi-open end, it further comprises a pair of lock catch windows 14 which are set on one end and are relatively arranged and butterfly wings 16 which are set on the other end and adjacent to the needle head 22. The open allows the access of the needle head 22, the barrier block 120 is arranged between the open and the lock catch block 122, and an inclined plane which allows the expansion arms pass through and makes the expansion arms gradually shrink is arranged between the barrier block 120 and the lock catch block 122.

The shape of the needle base 20 is matched with the sleeve 10, it further comprises a channel 21 which is connected with the inside of the needle head 22 and the lock catch head 28, and a lock catch head 28 which can be buckled with the lock catch window 14, the lock catch head 28 comprises a pair of buckling arms 280 which are buckled with the corresponding lock catch windows 14 and can be deformed elastically, and a through hole 282 which is connected with the conduit 30. The limiting part 24 is arranged between the needle head 22 and the locking part 26, which can prevent the needle base 20 separating from the sleeve 10. The tail end of the buckling arm 280 is provided with a convex part which is extending outwards, and is buckled on the inner wall of the lock catch window 14 in compressive deformation, and a gap with allowable elastic deformation is allowable between the buckling arm 280 and the lock catch head 28. Because the needle base 20 and the sleeve 10 are in elliptic cylinder and hole coordination, and also can adopt other shapes of coordination, which can effectively prevent the needle base 20 from rotating in the sleeve 10; the needle head 22 and the needle base 20 are fixedly connected, the lock catch head 28 needs to be held when the needle head 22 uses pump back, so the buckling arm 280 can be elastically deformed to separate from the coordination of the lack catch window 14 and release the lock catch head 28, and drive the needle base 20 and the needle head withdrawing backwards; and because the needle base 20 and the sleeve 10 are in elliptic cylinder and hole coordination, man-made rotation in operation can be prevented.

The invention avoids the cross infection between patients, and avoids the secondary use of medical needle and puncture of working staff after use or in the process of destroying, at the same time, this invention also can realize self-destruction and prevent man-made rotation of the medical needle.

Of course, the embodiment above only explains the technical considerations and characteristics of this invention, the purpose is to let people familiar with the technology understand the contents of the invention and implement thereby, which cannot limit the protection scope of this invention on this account. Equivalent conversion or modification according to the spiritual essence of main technical scheme of this invention should be covered in the protection scope of the invention.

1. A self-destructive type safe medical needle, comprising a sleeve (10) with an inner cavity (12), a needle base (20) which is arranged for reciprocating motion in the inner cavity (12) and has a needle head (22), a lock catch head (28) buckled with the needle base (20), and a conduit (30) connected with the inside of the lock catch head (28); the sleeve (10) is provided with a barrier block (120) and a lock catch block (122) in the inner cavity (12), and the needle base (20) is correspondingly provided with a limiting part (24) which is propped against and matched with the barrier block (120) and a deformable locking part (26) which is locked and matched with the lock catch block (122).

2. The self-destructive type safe medical needle according to claim 1, wherein the barrier block (120) prevents motion of the needle base (20), and the lock catch block (122) further prevents motion of the needle base (20).

3. The self-destructive type safe medical needle according to claim 2, wherein the locking part (26) is a pair of expansion arms which can be expanded elastically, a tail end of the expansion arm is propped and contacted with a plane of the lock catch block (122), and the barrier block (120) and the limiting part (24) are in contact.

4. The self-destructive type safe medical needle according to claim 1, 2 or 3, wherein the sleeve (10) further comprises a pair of lock catch windows (14) set on one end and butterfly wings (16) set on the other end.

5. The self-destructive type safe medical needle according to claim 4, wherein the lock catch head (28) is buckled with the lock catch window (14), the lock catch head (28) comprises a pair of buckling arms (280) which are buckled with the corresponding lock catch windows (14) and can be deformed elastically, and a through hole (282) which is connected with the conduit (30).

6. The self-destructive type safe medical needle according to claim 4, wherein the sleeve (10) is a hollow sleeve with one open end and one semi-open end, the barrier block (120) is arranged between the open end and the lock catch block (122), and an inclined plane which allows the expansion arms pass through and makes the expansion arms gradually shrink is arranged on the sleeve between the barrier block (120) and the lock catch block (122).

7. The self-destructive type safe medical needle according to claim 5, wherein the limiting part (24) is arranged between the needle head (22) and the locking part (26), a tail end of the buckling arm (280) is provided with a convex part which is extending outwards, and is buckled on the inner wall of the lock catch window (14) in compressive deformation, and a gap in elastic deformation is allowable between the buckling arm (280) and the lock catch head (28).

8. The self-destructive type safe medical needle according to claim 4, wherein a section of the inner cavity (12) is polygonal, elliptic or partially arc-shaped, and the section of the needle base (20) is circular, elliptic, polygonal or partially arc-shaped.
